(19) United States
(12) Patent Application Publication
(10) Pub. No.: US 2006/0127472 A1
(43) Pub. Date: Jun. 15, 2006

(54) Taste-masked prednisolone oral formulations

(76) Inventor: Keith Whitehead, Oldsmar, FL (US)

Correspondence Address: WYATT BARTON PRATT, 15 COLUMBIA AVENUE, HOPEWELL, NJ, 08525, US

(21) Appl. No.: 11/010,472

(22) Filed: Dec. 13, 2004

(51) Int. Cl. A61K 31/573; A61K 9/20

(52) U.S. Cl. 424/464; 514/179

(57) ABSTRACT

An improved taste-masked pharmaceutical prednisolone composition contains prednisolone sodium phosphate taste-masked with an effective taste-masking amount of rum ether.

### BACKGROUND OF THE INVENTION

1. Field Of The Invention

The present invention pertains to oral pharmaceutical compositions containing an active pharmaceutical ingredient of prednisolone taste-masked with rum ether.

2. Brief Description of the Related Art

Prednisolone oral formulations are prescribed pharmaceutical dosages used for treatment of endocrine disorders, rheumatic disorders, dermatologic diseases, allergic states, ophthalmic diseases, respiratory diseases, hematologic disorders, neoplastic diseases, edematous states, gastrointestinal diseases, nervous system conditions and other conditions. For example, prednisolone sodium phosphate is chemically known as pregna-1,4-diene-3,20-dione, 11,17-dihydroxy-21-(phosphonooxy)-, disodium salt, (11b). Prednisolone sodium phosphate is a known active pharmaceutical ingredient (API) in medication regulated by the U.S. Food & Drug Administration (FDA). Prednisolone sodium phosphate formulations are marketed in the United States by several companies.

However, prednisolone has a bitter taste which is difficult to moderate. As such, there is a need in the art to provide a superior taste-masked prednisolone oral dosage form. The present invention addresses this and other needs.

### SUMMARY OF THE INVENTION

The present invention includes an improved taste-masked pharmaceutical prednisolone composition that includes prednisolone taste-masked with an effective taste-masking amount of rum ether. In a preferred embodiment, the prednisolone includes an aqueous formulation and a sweetener of corn syrup. The composition is useful in numerous forms of the prednisolone, including refrigerated and non-refrigerated prednisolone pharmaceutical solutions and pediatric formulations.

Surprising it has been discovered that rum ether provides an extremely effective anti-bitter mask for oral prednisolone that is far superior to other anti-bitter masking compositions.

Other features, advantages and embodiments of the invention will become apparent to those of ordinary skill in the art by the following description, accompanying examples and appended claims.

### DETAILED DESCRIPTION OF THE INVENTION

The present invention includes a taste-masked prednisolone oral formulation. The formulation uses a taste-masking ingredient of rum ether to overcome the bitter taste of the prednisolone. It has been surprising discovered that rum ether provides an extremely effective anti-bitter mask for oral prednisolone, that is far superior to other anti-bitter masking compositions. Rum ether, also known as ethyl oxyhydrate, CAS number 8030-89-5/EC Number 232-449-5 is a synthetic flavoring agent which is stable, colorless to yellow liquid of ethereal rum-like note. It has a boiling point of between 65° C. and 87° C. and a density of 0.825 g/mL at 25° C. It has been known since the 1910s, and has been used as an artificial rum flavoring component, in beverages, candy and ice cream. Rum ether is available from Pharmaceutical Flavor Clinic, a division of Foote & Jenks, of Camden, N.J. under the tradename of PFC-9885 bitter mask, Sigma-Aldrich of Geneva, Switzerland and Penta Manufacturing Company of Livingston, N.J., USA. Rum ether is an aliphatic ester resulting from the destructive distillation of wood and ethyl alcohol. Components of rum ether are generally described as at least 99% water, ethyl alcohol, ethyl acetate, methanol, ethyl formate, acetone, acetaldehyde and formaldehyde, with methanol and formaldehyde contents not exceeding 5%. See, for example, Fenaroli's Handbook of Flavor Ingredients, 4^{th} Edition, edited by George A. Burdock, Ph.D., CRC Press, 2002, pp. 1601-1602 and Encyclopedia of Food and Color Additives, edited by George A. Burdock, Ph.D., Volume III, P-Z, CRC press 1997, pp. 2465-2467, the disclosure of both are herein incorporated by reference. Rum ether is preferably present in an amount of from about 0.001:1 to about 0.1:1 weight ratio amount of rum ether to prednisolone sodium phosphate, with a more preferred range of from about 0.01:1 to about 0.1:1 weight ratio of rum ether to prednisolone sodium phosphate. Effective amounts of anti-bitter mask component of rum ether may include, for example, from about 0.5 ml to about 20 ml of anti-bitter mask per 1 liter of solution, more preferably from about 1 ml to about 10 ml of anti-bitter mask per 1 liter of solution, and most preferably from about 5 ml to about 7.5 ml of anti-bitter mask per 1 liter of solution. The prednisolone composition of the present invention having the rum ether component may include any appropriate dosage form for the administration of prednisolone having the need for taste masking of the prednisolone active agent, such as chewable, aqueous and solid oral formulations, and combinations thereof. Of these, the aqueous oral formulation is best administered as a rum ether taste-masked active pharmaceutical ingredient. Representative aqueous formulations provide a liquid or other similarly created formulations for oral administration such as syrups, suspensions, solutions, and other like dosage forms. As described herein, the preferred embodiment the present invention includes prednisolone sodium phosphate and rum ether in combination with appropriate excipients for the administration of the prednisolone. The prednisolone formulations taste-masked with rum ether provide a novel system are well suited for use in either refrigerated or non-refrigerated prednisolone oral pharmaceutical formulations, and are particularly well suit for use in pediatric formulations.

The prednisolone composition of the present invention may include any appropriate dosage form for the administration of prednisolone having the need for taste masking of the prednisolone active agent, such as chewable, aqueous and solid oral formulations, and combinations thereof. Of these, the aqueous oral formulation is best administered as a taste-masked active pharmaceutical ingredient. Representative aqueous formulations provide a liquid or other similarly created formulations for oral administration such as syrups, suspensions, solutions, and other like dosage forms.

As described herein, the preferred embodiment the present invention includes prednisolone sodium phosphate and rum ether in combination with appropriate excipients for the administration of the prednisolone. Representative prednisolone formulations are found in U.S. patent application Ser. No. 10/742,332 to Whitehead, filed Dec. 18, 2003, entitled “Stabilized Prednisolone Sodium Phosphate Solutions”, the disclosure of which is herein incorporated by reference. The prednisolone formulations herein provide a novel system are well suited for use in either refrigerated or non-refrigerated prednisolone oral pharmaceutical formulations, and are particularly well suit for use in pediatric formulations.

These aqueous compositions or formulations of prednisolone sodium phosphate generally have uniformly dispersed mixtures at the molecular or ionic level of solute, which includes prednisolone sodium phosphate, rum ether, and appropriate excipients such as those detailed herein, mixed in a primary solvent of water. Pharmaceutically acceptable formulations of the present invention are conveniently prepared by adding an aqueous solution of prednisolone sodium phosphate together with the rum ether, with other excipients preferably added.

The aqueous solutions of the present invention include an appropriate amount of the active pharmaceutical agent of prednisolone, generally having an amount that is therapeutically effective in a convenient dosage unit for a given patient. Preferred amounts of prednisolone, preferably in the form of prednisolone sodium phosphate, contained within the pharmaceutical formulation of the present invention include, for example without limitation, up to about 50 grams of prednisolone sodium phosphate per liter of solution, with preferred ranges of from about 0.5 grams to about 40 grams of prednisolone sodium phosphate per liter of solution, more preferably from about 1 gram to about 25 grams of prednisolone sodium phosphate per liter of solution, and most preferably from about 5 grams to about 10 grams of prednisolone sodium phosphate per liter of solution. As the relative amount of prednisolone sodium phosphate is increased within a given volume of solution, such as over about 20 grams of prednisolone sodium phosphate per liter, the solution becomes increasing problematic to readily taste-mask. Rum ether is preferably present in an amount of from about 0.001:1 to about 0.1:1 weight ratio amount of rum ether to prednisolone sodium phosphate, with a more preferred range of from about 0.01:1 to about 0.1:1 weight ratio of rum ether to prednisolone sodium phosphate. Effective amounts of anti-bitter mask component of rum ether may include, for example, from about 0.5 ml to about 20 ml of anti-bitter mask per 1 liter of solution, more preferably from about 1 ml to about 10 ml of anti-bitter mask per 1 liter of solution, and most preferably from about 5 ml to about 7.5 ml of anti-bitter mask per 1 liter of solution.

The present invention may include suitable buffers (also referred to herein as “buffer salts” or “buffering agent”). As used herein, the term “buffers” is intended to include compounds used to resist a change in pH upon dilution or addition of acid or alkali. Representative buffering agents of the present invention include, without limitation, potassium dihydrogen orthophosphate, disodium hydrogen orthophosphate, disodium hydrogen orthophosphate, potassium phosphate, potassium phosphate monobasic, potassium phosphate dibasic, sodium phosphate monobasic, sodium phosphate dibasic, potassium metaphosphate, citric acid, monobasic sodium acetate and sodium citrate anhydrous and dehydrate and other such like materials known in the art, and combinations thereof. The amount of buffering agent possibly included in the formulation includes appropriate amounts as determinable by one skilled in the art of prednisolone oral formulations.

Additionally, a pH adjusting composition may be used to further adjust the pH of the composition, with pH adjusting compositions readily known in the art. Preferably, sodium hydroxide is used. Preferred pHs of the aqueous formulation of the present invention range from about 6.0 to about 8.5, such as for example from about 6.2 to about 8.2, particularly about 6.5 to about 7.2, more particularly about 6.6 to about 7.0, still more preferably about 6.7 to about 6.9, and most preferably about 6.8. Variations and adjustments of the pH of the aqueous formulation is preferably obtained by moderating the addition of the buffer salt(s) and pH adjusting composition(s).

Solutions of the present invention may include an appropriate amount of thickening agent (also referred to herein as “viscosity enhancing agents), effective to stabilize the prednisolone sodium phosphate component within aqueous composition, in combination with the buffering agent. Representative thickening agents of the present invention include, for example without limitation, cellulose derivatives such as carboxymethylcellulose or a salt thereof of a C₁₋₄ alkyl and/or a hydroxy-C₂₋₄ alkyl ether of cellulose, such as methylcellulose, ethylcellulose, hydroxyethylcellulose, hydroxymethylcellulose, hydroxypropylcellulose, hydroxyethylmethylcellulose and hydroxypropylmethylcellulose. Preferably the thickening agent comprises a hydroxy-cellulose derivative, and most preferably the thickening agent comprises hydroxyethylcellulose. When present, preferably the cellulose derivative is present in an amount of up to about 50 grams of cellulose derivative per liter of solution, with preferred ranges of from about 0.5 grams to about 40 grams of cellulose derivative per liter of solution, more preferably from about 1 gram to about 25 grams of cellulose derivative per liter of solution, and most preferably from about 1.5 grams to about 10 grams of cellulose derivative per liter of solution.

The prednisolone sodium phosphate solutions of the present invention may further include known excipients for pharmaceutical formulations, as appropriate, including preservatives, coloring agents, sweetening agents, flavoring agents, additional anti-bitter mask components, etc. Representative preservatives suitable for use in the present invention include, for example without limitation, one or more alkyl hydroxybenzoates, such as methyl hydroxybenzoates, ethyl hydroxybenzoates, propyl hydroxybenzoates, butyl hydroxybenzoates and the like. Additional preservatives useful in the present invention include, but are not limited to, sodium benzoate, potassium sorbate, salts of edetate (also know as salts of ethylenediaminetetraacetic acid, or EDTA, such as disodium edetate) and antimicrobial agents including parabens (p-hydroxybenzoic acids esters) such as methylparaben, ethylparaben, propylparaben, butylparaben and the like. The preservatives listed herein are exemplary, with the appropriate preservative and amount of a preservative incorporated into the solution as determinable by one skilled in the art for compatibility and efficacy of the preservative in a given solution. Techniques and methods for evaluating preservative efficacy in a given pharmaceutical formulations are readily known in the art. Parabens are preferred, with methylparaben most preferred for use as preservative ingredients to add to the present pharmaceutical solution, although other pharmaceutically acceptable preservatives may be substituted therefor. Preservatives may be included in a given pharmaceutical formulation of the present invention as appropriate, with preferred amounts of up to 1 gram per 100 mL of the solution. More preferably the preservatives are included in amounts that range of from about 0.10 to about 0.75 grams per 100 mL of the solution, still more preferably from about 0.15 to about 0.5 grams per 100 mL of the solution, and most preferably from about 0.20 to about 0.4 grams per 100 mL of the solution.

Coloring agents also may be incorporated in the solution of the present invention as determined by one skilled in the art to be appropriate, for chemical compatibility with other ingredients in the solution and the like. Coloring agents are generally used to provide an appealing color to the solution. Suitable coloring agents for use in pharmaceutical solutions are well known in the art. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and iron oxide (black, red, yellow), other F.D. & C. dyes and natural coloring agents such as grape skin extract, beet red powder, beta-carotene, annato, carmine, turmeric, paprika, combinations thereof and other such materials known to those skilled in the art.

The pharmaceutical formulation of the present invention preferably contains flavoring agents (herein referred to also as “flavorants”), sweetening agents, and combinations thereof to mask the inherently bitter taste associated with prednisolone, and thereby improving the palatability of the solution of the present invention. Flavorants are used to impart a pleasant flavor and often odor to a pharmaceutical preparation. Suitable flavoring agents include natural and artificial flavors, such as synthetic flavor oils and flavoring aromatics and/or natural oils, extracts from plants, leaves, flowers, fruits and so forth and combinations thereof. Representative suitable flavoring agents may be for example, without limitation, menthol, cinnamon, wintergreen, clove, bay, anise, eucalyptus, thyme, cedar leave, nutmeg, sage, bitter almonds and cassia, vanilla, artificial vanilla, chocolate, artificial chocolate, bubble gum, both natural and artificial fruit flavors, such as cherry flavor, grape flavor, orange flavor, strawberry flavor, lemon flavor, grapefruit flavor and “mint” flavors such as peppermint flavor and spearmint flavor, lime flavor, apple flavor, pear flavor, peach flavor, raspberry flavor, plum flavor, pineapple flavor, apricot flavor and so forth, including combinations of two or more thereof. Flavoring agents are generally provided as a minor component of the solution in amounts effective to provide a palatable flavor to the solution. The amount of flavoring agent may depend on a number of factors, including the desired organoleptic effect. The precise amount of sweetening and/or flavoring agent(s) depends on the properties of the agent(s) used, however generally in an amount that is sufficient to mask the bitter taste associated with prednisolone as determinable by one skilled in the art. However, flavoring agents are generally present in the solution in amounts in the range of from about 0 grams to about 10 grams per 100 mL of the solution, with preferred amounts of from about 2 grams to about 5 grams per 100 mL. Sweeteners suitable for inclusion in the present invention may be determined by one skilled in the art including, for example without limitation, both natural and artificial sweeteners such as the representative sweetening agents of intense sweeteners such as sorbitol, sucrose, saccharins such as sodium saccharin, cyclamates such as sodium cyclamates, aspartame, sucralose, thaumatin, acesulfam K, and the like, and sugars such as monosaccharides, disaccharides and polysaccharides. Representative sugars useful in the present invention include, without limitation, xylose, ribose, glucose, mannose, galactose, fructose, dextrose, sucrose, maltose, partially hydrolyzed starch or corn syrup, and sugar alcohols such as sorbitol, xylitol, mannitol, glycerin, etc. and combination thereof. Presently preferred sweetener includes partially hydrolyzed starch or corn syrup. The amount of sugar sweetener used in the solution varies with the degree of sweetening desired for the particular formulation as determinable by one skilled in the art, with preferred amounts of sugar sweetener ranging from about 0 grams to about 100 grams sugar sweetener per 100 mL of the solution, more preferably from about 20 grams to about 95 grams per 100 mL of solution, still more preferably from 30 grams to about 90 grams sugar sweetener per 100 mL of the solution, and most preferably from about 40 grams to about 85 grams per 100 mL of solution. Sugar sweeteners may be replaced or augmented by water soluble artificial sweeteners, such as the suitable artificial sweeteners previously listed and mixtures thereof. The amount of artificial sweetener used in the solution may vary to provide an appropriate amount of sweetness to the solution as determinable by one skilled in the art, generally in amounts similar to those of sugar sweeteners described above. Mixtures of sweetening and/or flavoring agents are preferably used.

In one preferred embodiment of the present invention as an oral formulation, the aqueous formulation may comprise prednisolone sodium phosphate dissolved in purified water, an effectively stabilizing amount of rum ether, a preservative and sweetener, with the pH of the aqueous formulation moderated by the use of appropriate pH adjusting compositions. More preferably, the solution also contains other conventional excipients such as flavoring aids.

The present invention provides methods of treating a subject (e.g., mammal, particularly humans) comprising administering to a subject in need of such treatment a therapeutically effective amount of at least one active ingredient, formulation thereof, or unit dose forms thereof, each as described herein. As used herein, the term “treatment”, or a derivative thereof, contemplates partial or complete inhibition of the stated disease state such as, for example, endocrine disorders, rheumatic disorders, dermatologic diseases, allergic states, ophthalmic diseases, respiratory diseases, hematologic disorders, neoplastic diseases, edematous states, gastrointestinal diseases, nervous system conditions and other conditions, when an active ingredient of the present invention is administered prophylactically or following the onset of the disease state for which such active ingredient of the present invention is administered. For the purposes of the present invention, “prophylaxis” refers to administration of the active ingredient(s) to a mammal to protect the mammal from any of the disorders set forth herein, as well as others.

The typical active daily dose of the prednisolone sodium phosphate depends on various factors such as, for example, the individual requirement of each patient, the route of administration, and the disease. An attending physician may adjust the dosage rate based on these and other criteria if he or she so desires. As an example, a suitable oral dosage form may encompass from about 1 mg to about 35 mg total daily dose, typically administered in one single dose or equally divided doses. A more preferred range is from about 5 mg to about 25 mg total daily dose, and a most preferred range is from about 10 mg to about 20 mg total daily dose, such as about 15 mg. It should be appreciated that daily doses other than those described above may be administered to a subject, as appreciated by an attending physician. Within this dosage, the rum ether is preferably present in an amount of from about 0.00025 mL to about 0.01 mL of rum ether.

The phrase “pharmaceutically acceptable” is employed herein to refer to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The amount of therapeutic compound incorporated in each device of the present invention will be at least one or more dosage form and can be selected according to known principles of pharmacy. An effective dosage of therapeutic compound is specifically contemplated. By the term “effective dosage”, it is understood that, with respect to, for example, pharmaceuticals, a pharmaceutically effective amount is contemplated. A pharmaceutically effective dosage is the amount or quantity of a drug or pharmaceutically active substance which is enough for the required or desired therapeutic response, or in other words, the amount, which is sufficient to elicit an appreciable biological response when, administered to a patient. The appreciable biological response may occur as a result of administration of single or multiple unit doses of an active substance. Depending upon the active substance used and upon the amount of active substance present in a particular device according to the present invention, a unit dose may comprise one or more such devices.

### Rum Ether Taste-Masked Formulation

An aqueous pharmaceutical prednisolone composition of the present invention was formulated by preparing a mixture of hydroxyethylcellulose dissolved in 500 milliliters of purified water with 5 mL of rum ether, with potassium phosphate dibasic and potassium phosphate monobasic added (from a hot water mixture). 4.0 grams of prednisolone sodium phosphate were then added and mixed until dissolved. Sodium hydroxide was added to adjust the pH to from about 6.7 to about 6.9. The mixture was filled to a quantity of 1 liter with purified water. The taste of the solution was significantly improved over ten known flavored prednisolone sodium phosphate formulations not using rum ether.

The foregoing summary, description, and examples of the invention are not intended to be limiting, but are only exemplary of the inventive features which are defined in the claims.

What is claimed is:

1. An improved taste-masked oral pharmaceutical prednisolone composition comprising prednisolone taste-masked with an effective taste-masking amount of rum ether.

2. The taste-masked composition of claim 1, wherein the composition is selected from the group consisting of chewable, aqueous and solid oral formulations, and combinations thereof.

3. The taste-masked composition of claim 2, wherein the composition comprises an aqueous oral formulation.

4. The taste-masked composition of claim 1, wherein the prednisolone comprises prednisolone sodium phosphate.

5. The taste-masked composition of claim 3, wherein the rum ether is present in an amount of from about 0.5 mL to about 20 mL per 1 liter of solution.

6. The taste-masked composition of claim 5, wherein the rum ether is present in an amount of from about 1 mL to about 10 mL per 1 liter of solution.

7. The taste-masked composition of claim 6, wherein the rum ether is present in an amount of from about 5 mL to about 7.5 mL per 1 liter of solution.

8. The taste-masked composition of claim 1, wherein the rum ether is present in an amount of from about 0.001:1 to about 0.1:1 weight ratio amount of rum ether to prednisolone sodium phosphate.

9. The taste-masked composition of claim 8, wherein the rum ether is present in an amount, of from about 0.01:1 to about 0.1:1 weight ratio amount of rum ether to prednisolone sodium phosphate.

10. The taste-masked composition of claim 3, further comprising a sweetener.

11. The taste-masked composition of claim 10, wherein the sweetener is selected from the group consisting of corn starch, sorbitol, sucrose, saccharins, cyclamates, sugars and combinations thereof.

12. The taste-masked composition of claim 11, wherein the sweetener includes partially hydrolyzed starch or corn syrup.

13. The taste-masked composition of claim 12, wherein the partially hydrolyzed starch or corn syrup is present in an amount of from about 20 to about 95 grams per 100 mL of the total composition.

14. The taste-masked composition of claim 13, wherein the corn starch is present in an amount of from about 30 to about 90 grams per 100 mL of the total composition.

15. The taste-masked composition of claim 1, further comprising an adjunct component selected from the group consisting of preservatives, parabens, flavoring agents and combinations thereof.

16. A refrigerated prednisolone pharmaceutical solution comprising the composition of claim 1.

17. A non-refrigerated prednisolone pharmaceutical solution comprising the composition of claim 1.

18. A unit dosage of the composition of claim 1, wherein the prednisolone sodium phosphate is present in an amount of from about 5 mg to about 25 mg of prednisolone sodium phosphate.

19. A unit dosage of the composition of claim 1, wherein the rum ether is present in an amount of from about 0.00025 mL to about 0.01 mL of rum ether.

20. A pediatric pharmaceutical prednisolone composition of claim 1.
